(19) United States
(12) Patent Application Publication
(10) Pub. No.: US 2024/0366196 A1
(43) Pub. Date: Nov. 7, 2024

(54) BRUSH FOR BIOPSY

(71) Applicant: ORLANDO HEALTH, INC., Orlando, FL (US)

(72) Inventors: Yamen Smadi, Orlando, FL (US); Devendra Indulal Mehta, Orlando, FL (US); Chirajyoti Deb, Sanford, FL (US); Anthony Mango, Orlando, FL (US); Riham Mikhail Hanna, Winter Park, FL (US); Evan Cuellar Gomez, Orlando, FL (US)

(73) Assignee: ORLANDO HEALTH, INC., Orlando, FL (US)

(21) Appl. No.: 18/777,241

(22) Filed: Jul. 18, 2024

Related U.S. Application Data
(63) Continuation-in-part of application No. 18/341,591, filed on Jun. 26, 2023.
Application No. 18/341,591 is a continuation of application No. 16/845,607, filed on Apr. 10, 2020, now Pat. No. 11,723,631.

(51) Int. Cl. A61B 10/02

(52) U.S. Cl. CPC A61B 10/02; A61B 2010/0216

(57) ABSTRACT

A brush for biopsy may include a sheath and a shaft extending through a cavity of the sheath, where the shaft is moveable relative to the sheath. The brush may include a cap located at a distal end of the shaft, where the cap is configured to seal a distal end of the sheath. The brush may further include a set of bristles and at least two members attached to the shaft at locations proximal to the cap, where the at least two members are configured to seal the cavity when located within the sheath. A first member of the at least two members may be attached to the shaft at a location distal to the set of bristles, and a second member of the at least two members may be attached to the shaft at a location proximal to the set of bristles.

### RELATED APPLICATION(S)

This application is a continuation-in-part of U.S. patent application Ser. No. 18/341,591, filed Jun. 26, 2023, entitled “BRUSH FOR NON-INVASIVE BIOPSY,” which is a continuation of U.S. patent application Ser. No. 16/845,607, filed Apr. 10, 2020, entitled “BRUSH FOR NON-INVASIVE BIOPSY” that issued as U.S. Pat. No. 11,723,631 on Aug. 15, 2023. The applications and patent listed in this paragraph are hereby incorporated by reference in their entirety.

### TECHNICAL FIELD

The present disclosure relates to a brush, or tissue sample collecting device, for invasive (with sedation) or non-invasive (without sedation) biopsy or other types of sample collections via endoscopy and/or colonoscopy and any other related methods of use.

### BACKGROUND

Many esophageal diseases require serial evaluation of the esophageal epithelium by endoscopy. Nasoesophageal brushes allow medical professionals to obtain pan-esophageal epithelial specimens in an efficient, sedation-free, and office-based setting. Such devices are intended to compliment endoscopy. Once in the esophagus, a brush can gently collect a tissue sample from the esophageal epithelium within the brush itself and in a specific compartment. Tests may then be run on collected samples to evaluate diseases.

In one example, eosinophilic esophagitis (EoE) is a chronic, immune mediated disease characterized clinically by symptoms of esophageal dysfunction and histologically by eosinophil-predominant inflammation. EoE has become a major cause of upper gastrointestinal morbidity in children and adults, with an estimated prevalence between 25.9 and 56.7/100,000 persons in the United States.

Because clinical symptoms provide only a modestly accurate prediction of inflammation in these patients, visual evaluation and biopsies via endoscopy remain the standard methods to monitor disease activity. However, this methodology is challenging, especially in patients who manage the disease via dietary elimination, because an average of 6-11 endoscopies under general anesthesia are required to identify dietary triggers. Nasoesophageal brushes may be used after food challenges instead of endoscopy. Further, the disease is patchy and the evaluation by means of light microscopy after hematoxylin and eosin staining of small biopsies might not accurately reflect eosinophil involvement due to degranulation. Other exemplary diseases that may be evaluated via nasoesophageal brushes include candida esophagitis, infectious esophagitis, and esophageal cancer.

Other common diseases, like small intestinal bacterial overgrowth or small intestinal microbial overgrowth, may occur elsewhere in the gastrointestinal tract or in colorectal locations and also require evaluation and/or monitoring via endoscopy and/or non-invasive or invasive (via endoscopy or colonoscopy) biopsies. Such diseases may exist beyond the esophagus in the small intestine, including in the duodenum, jejunum, and ileum, and in the large intestine, colon, and other colorectal locations. Evaluating and monitoring these diseases often require similar collection of tissue samples, including samples from epithelial and/or mucosal layers of the gastrointestinal tract, for example. While collecting tissue samples, it is pertinent to collect samples from precise locations within the esophagus, other upper and lower gastrointestinal tract locations, the colon, and other colorectal locations. It is also important to avoid sample contamination from gastrointestinal fluids and other contamination sources in the upper and lower gastrointestinal tract and from other colorectal locations, which may negatively affect testing of the collected tissue samples.

Brushes, or tissue sample collection devices, for collecting tissue samples (including gastrointestinal tract and/or mucosal surface samples) to evaluate and monitor esophageal, gastrointestinal, and colorectal diseases may be compatible, such as sized and/or shaped, for use with conventional endoscopes, including gastroscopes and colonoscopes, and also for transnasal endoscopy (TNE). One example of a transnasal gastroscope is the EvoEndo® Model LE Single-Use Gastroscope.

In view of the above, there is a critical need for a less invasive, more accurate, and more economical method to monitor and evaluate esophageal, gastrointestinal, and colorectal diseases. The embodiments of a brush, or a tissue sample collection device, for biopsy discussed below address these issues.

### BRIEF DESCRIPTION OF THE DRAWINGS

The embodiments discussed herein may be better understood with reference to the following drawings and description. The components in the figures are not necessarily to scale. Moreover, in the figures, like-referenced numerals designate corresponding parts throughout the different views.

FIG. 1 is an illustration showing a brush for biopsy during deployment to a location in the esophagus in accordance with certain aspects of the present disclosure.

FIG. 2 is an illustration showing the brush of FIG. 1 in an operational state such that a distal end of the brush engages a target tissue area in the esophagus in accordance with certain aspects of the present disclosure.

FIG. 3 is an illustration showing a sectional view of the brush from FIG. 1 in a first (deployment) state in accordance with certain aspects of the present disclosure.

FIG. 4 is an illustration showing a sectional view of the brush from FIG. 1 in a second (operational) state in accordance with certain aspects of the present disclosure.

FIG. 5 is an illustration showing another embodiment of a brush for biopsy in a first (deployment) state, where a distal tip of the brush is fixed relative to a set of bristles in accordance with certain aspects of the present disclosure.

FIG. 6 is an illustration showing the brush of FIG. 5 in a second (operational) state in accordance with certain aspects of the present disclosure.

FIG. 7 is an illustration showing another embodiment of a brush for biopsy in a first (deployment) state, where a distal tip is movable relative to an elongated sheath and also a set of bristles in accordance with certain aspects of the present disclosure.

FIG. 8 is an illustration showing the brush of FIG. 7 in a second (operational) state in accordance with certain aspects of the present disclosure.

FIG. 9 is an illustration showing another embodiment of a brush for biopsy in a first (deployment) state, where a shaft including a cap, a set of bristles, and at least two members is movable relative to an elongated sheath in accordance with certain aspects of the present disclosure.

FIG. 10 is an illustration showing the brush of FIG. 9 in a second (operational) state in accordance with certain aspects of the present disclosure.

### DETAILED DESCRIPTION

The present disclosure relates to a brush, or a tissue sample collection device, for non-invasive biopsy and methods of use. The following embodiments, and variations thereof, provide a less invasive, more accurate, and more economical method for monitoring certain diseases in the human or animal body. For example, the brush embodiments described herein may provide the ability to perform a relatively non-invasive or minimally invasive, accurate, and economical method for obtaining a biopsy sample from within the esophagus and gastrointestinal tract for evaluating and monitoring EoE and other gastrointestinal diseases. Further, unlike other methodologies, the present embodiments may be deployed through the nose and nasal cavity, or via a gastroscope, to a target location and then operated from a location outside the body (e.g., at the device's proximal end outside of the body).

While the specific embodiments described below are tailored towards deployment through the nose for engagement with tissue inside the esophagus and gastrointestinal tract, other uses are also contemplated. For example, the non-limiting embodiments described below, and variations thereof, may be used for engagement with any suitable tissue area within a human or animal body.

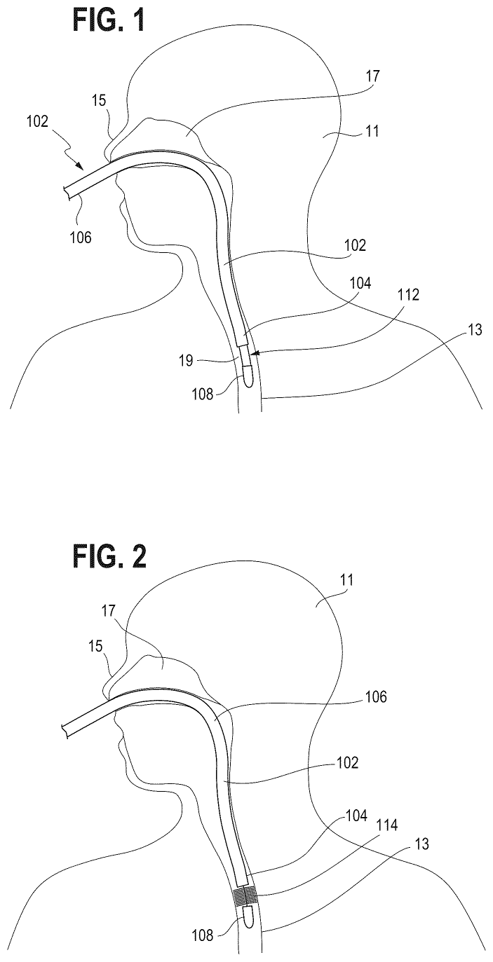

FIGS. 1-2 show a non-limiting example of a brush 102 for biopsy. As discussed above, while any suitable use of the brush 102 may be contemplated, the brush 102 may be specifically configured for collecting a tissue sample within the esophagus 13 (e.g., in performance of an esophageal biopsy during evaluating and monitoring of eosinophilic esophagitis). The brush 102 may include a sheath (e.g., a tube or other elongated device with a cavity therein) that extends the majority of the length of the brush 102. Deployment of the brush 102 to a target site within the esophagus 13 (or another location) may occur by inserting at least the distal end 104 of the brush 102 through the nose 15 and nasal cavity 17, for example. The sheath 106 and components therein may include sufficient flexibility (or another shape-related feature, such as a shape-memory metal) such that it can be guided through the nose 15 and the nasal cavity 17 and into the esophagus 13 without causing damage the surrounding tissue. For example, the tube may be formed of silicon, plastic, or another suitable material.

As shown in FIG. 1, the brush 102 may be deployed in a compacted first state (or deployment state), where the dimensions of the distal end 104 of the brush 102 are relatively small, and where certain components with deformities or discontinuities (e.g., bristles, as discussed below) are sealed within an outer casing such that they do not interfere with the brush's initial deployment. For example, the sheath 106 that extends to a distal end 104 of the brush 102 may primarily form the outer surface areas that contact body tissue when the brush 102 is in the depicted first state.

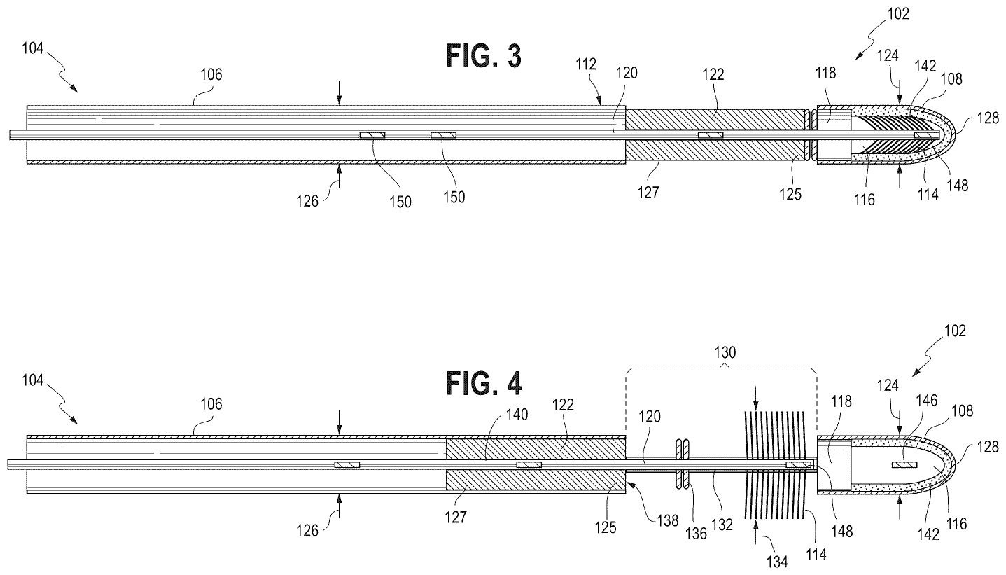

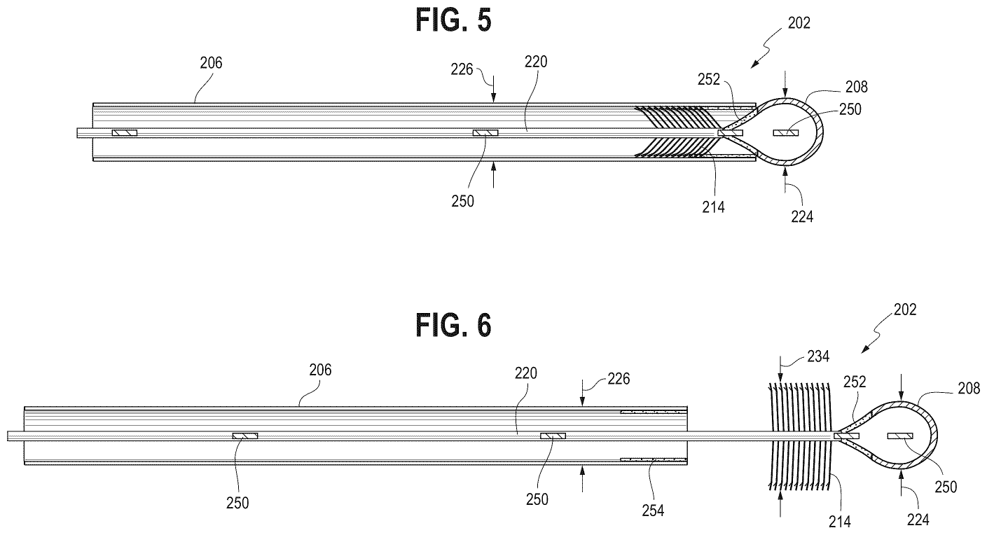

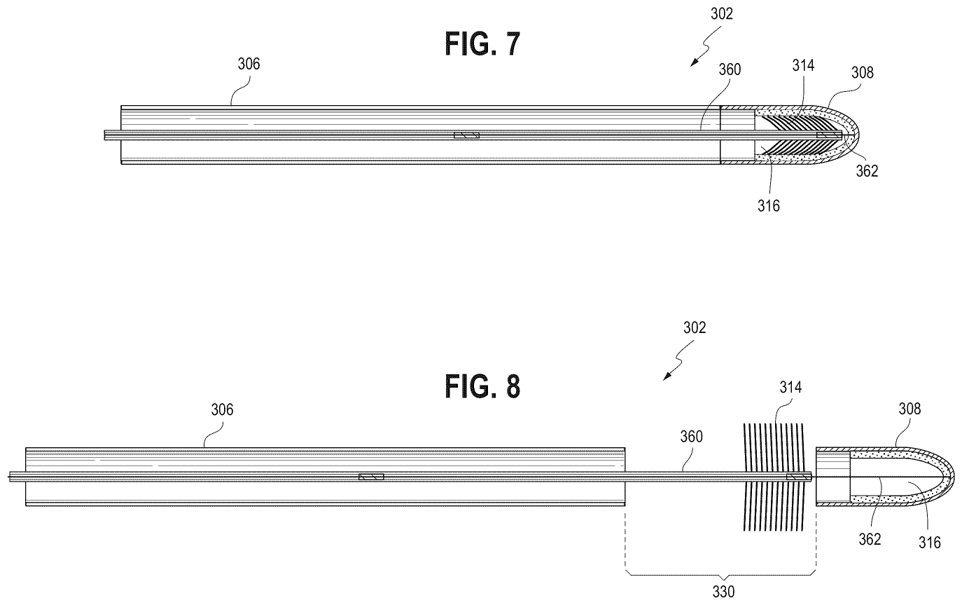

The distal end 104 of the brush 102 may include the distal tip 108, which may have a taper, a point, and/or other suitable feature for guiding the distal end 104 of the brush 102 to a target location. For example, the taper of the distal tip 108 may act to displace fluids, body tissue, and other particles from the movement path of the brush 102 as the brush 102 moves distally through the body. As discussed below, the distal tip 108 may be fixed to the sheath 106 (e.g., consistent with the embodiment of FIGS. 3-4), or it may be movable relative to the sheath 106 (e.g., consistent with the embodiment of FIGS. 5-6 and the embodiment of FIGS. 7-8). When in the first state of FIG. 1, the outer surface 112 of the sheath 106, distal tip 108, and remainder of the distal end 104 of the brush 102 may be substantially smooth and continuous (e.g., substantially lacking exposed discontinuities on the outer surface) to minimize friction between the brush 102 and surrounding tissue and to prevent damage to said tissue.

When the distal end 104 of the brush 102 is in an appropriate position, the brush 102 may be adjusted to an operational second state shown in FIG. 2. In this second state, certain working features, such as the depicted bristles 114, may be exposed within the body such that they are capable of engaging a target area 19 of body tissue. While not shown in FIGS. 1-2, the brush 102 may include features on its proximal end for controlling adjustment from the first state to the second state, and/or for controlling the working features at the distal end 104 of the brush 102. For example, at least one movable shaft (discussed below) may be engageable by a medical professional on the proximal end of the device. Advantageously, the working components on the distal end of the brush 102 may be operated from a location outside the patient body 11, thereby providing a procedure that is relatively non-invasive or minimally invasive relative to other procedures.

FIGS. 3-4 are illustrations depicting a sectional view of the distal end 104 of the brush 102 in the first state and second state, respectively. Referring to FIG. 1, a distal tip 108 of the brush 102 may include a cavity 116, where an opening 118 of the cavity 116 faces proximally. A shaft 120 may be included, which may be fixed to the bristles 114 such that when the shaft 120 moves, the bristles 114 also move. The shaft 120 (and therefore bristles 114) may be movable relative to the sheath 106 and also the distal tip 108. Like the sheath 106, the shaft 120 may be made of a material with sufficient flexibility such that it can bend in an appropriate manner as the distal end 104 of the brush 102 moves through curves within a body cavity. The same is true of other components extending through the sheath 106, if applicable.

Advantageously (and as mentioned above), the brush 102 may be relatively compact in the deployment state (or first state) of FIG. 3, which may facilitate passage through a body cavity during deployment to a target location (e.g., for the evaluation of mucosal inflammation in the esophagus, for example). In this first state, the bristles 114 may be located within the cavity 116 of the distal tip 108 such that they do not interfere with the deployment of the brush 102 as the brush 102 moves through the body towards a target area. In other words, the bristles 114 may be shielded from surrounding body tissue during deployment. To facilitate movement, the outer surface 112 of the brush 102 (including the distal tip 108, the sheath 106, etc.) may be smooth and have a relatively consistent cross-sectional shape and dimension to reduce friction between the outer surface 112 and surrounding body tissue. Further, the outer surface 112 may be substantially continuous (e.g., without discontinuities) to prevent moisture, body tissue, and other unwanted materials from collecting in discontinuities during the deployment procedure.

Further, to provide a substantially continuous outer surface of the brush 102 during deployment, an optional plug 122 may be included. The plug 122 may occupy space that will later be used as working space for movement of the bristles 114 (as discussed below). As shown in FIG. 3, a distal side 125 of the plug 122 may be adjacent to (and potentially in contact with) the distal tip 108, and a proximal side 127 of the plug 122 may be adjacent to (and potentially in contact with) the sheath 106. While not shown in this example, the plug 122 may at extend to a location within the sheath 106 and/or the distal tip 108 in the first state to ensure sufficient contact and/or sealing of the device.

A maximum diameter 124 of the distal tip 108 may be at least as large as an outer diameter 126 of the sheath 106. For example, the maximum diameter 124 of the distal tip 108 may be about the same size as the outer diameter 126 of the sheath 106 (at least at the distal end of the sheath 106). Other areas of the distal tip 108 may have a diameter (or other cross-sectional dimension) that is smaller than the outer diameter 126 of the sheath 106. For example, the distal tip 108 may taper or otherwise reduce its cross-sectional dimension as it moves towards an apex 128 at its distal terminus. This may be advantageous for guiding the brush 102 as it moves through the body, displacing objects in front of the brush 102 to facilitate smooth motion, etc.

To adjust the brush 102 from the first state (of FIG. 3) to the second state (of FIG. 4), the shaft 120 may be retracted in the proximal direction relative to the sheath 106 (which may move the plug 122 to create working space 130 for the bristles 114, as discussed below). Since the sheath 106 is fixed relative to the distal tip 108 (e.g., via a connector 132, which may extend from the sheath 106 to the distal tip 108), such movement also causes the shaft 120 to move in the proximal direction relative to the distal tip 108. When the bristles 114 are fixed to the shaft 120, such action may move the bristles 114 out of the cavity 116 of the distal tip 108 and into the working space 130. A medical professional can then control the movement of the bristles 114 by moving the shaft 120 back-and-forth (e.g., distally and proximally) such that the bristles 114 rub against a target tissue area. Body tissue may thereby be collected by the bristles 114.

As shown, the bristles 114 may include a tendency to expand to a brushing diameter 134 (or uninhibited operational diameter) when the brush 102 is in the second state (of FIG. 4). The brushing diameter 134 of the bristles 114 is defined as the uninhibited diameter of the bristles 114 (i.e., absent contact with a target tissue or another object) when the bristles 114 are located outside the cavity 116 of the distal tip 108. To measure the brushing diameter 134 of the bristles 114, the bristles 114 shall be allowed to expand to their default, uninhibited state at a location outside of a patient body at atmospheric pressure. The brushing diameter 134 is preferably larger the maximum diameter 124 of the distal tip 108 and the outer diameter 126 of the sheath 106. For example, the brushing diameter 134 may be at least 10% larger, such as at least 20% larger, such as at least 30% larger (or more) than the outer diameter of the sheath 106 (and/or the maximum diameter 124 of the distal tip 108. This relative sizing allows the bristles 114 to reach beyond the outer diameter of the sheath 106 such that a target tissue area can be adequately engaged by the bristles 114 for biopsy.

When the plug 122 is included, the shaft 120 may be fixed to a projection 136 that is configured (e.g., sized, positioned, and shaped) to contact a distal surface 138 of the plug 122 when the shaft 120 initially moves in the proximal direction. This contact may cause the plug 122 to move proximally as the shaft 120 moves proximally, resulting in the plug 122 moving into the sheath 106. Advantageously, such movement may provide working space 130 where the bristles 114 may operate. Further, the plug 122 may seal the distal-end opening of the sheath 106 such that it remains free from body fluids and/or other particles. While not shown, the shaft 120 may be fixed to another projection or similar structure, or alternatively a separate device may be included within the sheath 106, for pushing the plug 122 distally into its deployment position after a brushing process is complete.

The plug 122 may include a central channel 140 that is configured (e.g., sized and shaped) for receipt of the shaft 120. Thus, the shaft 120 may extend through the central channel 140. Further, it is contemplated that the plug 122 may be formed from a compressible material such that the central channel 140 compresses around the shaft 120 when the plug 122 is located inside the sheath 106, which may provide a desirable friction on the shaft 120. Advantageously, such friction may enhance the feel to a medical professional and increase the precision of movement when operating the bristles of the brush 102 (e.g., by adding a suitable amount of resistance to the shaft 120).

Optionally, the distal tip 108 may include tissue collection member 142. Without limitation, the tissue collection member 142 may include an absorbent lining or other absorbent feature (such as a sponge-like material or another feature with a characteristic suitable for collecting a tissue sample from the bristles 114), an adherent surface, one or more cavities for receiving and storing body tissue, etc. The tissue collection member 142 may be formed of any suitable material, which may include sufficient absorbency and/or another means of adhering to body tissue and/or fluid in accordance with this description, and it may be used to collect/retain samples which can measured in with any suitable method. For example, the tissue collection member 142 may include a pad of material that absorbs and stabilizes a tissue sample for further processing. Such pads would have a preservative aspect to prevent denaturing of proteins (as understood in the art).

For example, as shown in FIG. 3 (e.g., when the brush 102 is in the first state), the bristles 114 contact a tissue collection member 142 that is located inside the cavity 116 of the distal tip 108. Once the brush 102 is removed from a patient, the collected tissue samples may be obtained from the tissue collection member 142 (and/or the tissue collection member 142 may be removed and evaluated). In certain embodiments, it is contemplated that the tissue collection member 142 may include one or more properties that change based on the composition of the collected tissue sample. For example, the tissue collection member 142 may be configured to react to the presence of a certain biomarker or other chemical, where such reaction provides a visual indication to providing information to the medical professional (e.g., by changing colors).

The brush 102 may include one or more tracking devices and/or radiopaque markers configured to indicate a location of the distal tip 108 and/or the devices and/or markers within the brush 102. Without limitation, suitable tracking devices and/or radiopaque markers may include magnets, phantoms, and/or magnetic, metallic, and/or other suitable radiopaque material members that are identifiable in real-time via radiology (e.g., medical imaging such as x-ray, ultrasound, MRI, CT, or the like) and/or another type of tracking system. Such devices and/or markers, like magnets, are known by persons of ordinary skill in the art to be identifiable in various types of medical imaging systems. For example, such tracking devices and/or radiopaque markers may be formed from hard, soft, and digital materials that appear in medical imaging. In a non-exhaustive list, the materials for first tracking devices and/or radiopaque markers, such as phantoms, may include plastics, salt solutions, silicones, epoxy, polyurethane foams, carbon powder, water, disposable diapers, and radioactive substances. The tracking devices and/or radiopaque markers may be included as small members or pieces inserted into or otherwise affixed to structures for tracking (such as the shaft 120 or other structures). In other embodiments, the tracking devices and/or radiopaque markers may include a tape, paint, or other thin layer of substance adhered to the structure for tracking. Other approaches and example tracking devices and/or radiopaque markers are also contemplated. Referring to FIG. 4 for example, a first tracking device and/or radiopaque marker 146 may be located at the distal tip 108, and a second tracking device and/or radiopaque marker 148 may be located on the shaft 120. Beneficially, since the shaft 120 is fixed to the bristles 114, such an embodiment may provide real-time information to a medical professional regarding the relative positions of the bristles 114 and the distal tip 108, which may aid in a biopsy procedure. Additional tracking devices and/or radiopaque markers 150 may be included at any suitable location for verification purposes, for detection by more than one type of instrument, etc.

FIGS. 5-6 shows another embodiment of a brush 202 for collecting a tissue sample from a patient body. The brush 202 is similar to the embodiment shown in FIGS. 3-4, but it lacks a distal tip having a cavity for receiving a set of bristles 214, and its distal tip 208 is fixed to a shaft 220 that is also fixed to a set of bristles 214. For example, the distal tip 208 shown in FIGS. 5-6 is located at a distal end of a shaft 220. The bristles 214 are located on the shaft 220 (e.g., also fixed relative to the shaft) at a location that is proximal of the distal tip 208. Accordingly, the securement area 221 between the distal tip 208 and the shaft 220 may be fixed relative to where the bristles 214 are secured to the shaft 220.

In the depicted embodiment, a sheath 206 is movable at least in a proximal direction relative to the distal tip 208. The bristles 214 may be at least partially located in the sheath 206 when the brush 202 is in a first state or deployment state (e.g., shown in FIG. 5). The bristles 214 may include a tendency to expand to a brushing diameter 234 when the sheath 206 is in a second state (or operational/brushing state), as shown in FIG. 6. When released from the sheath 206 a brushing diameter 234 of the bristles 214 may be larger than an outer diameter 226 of the sheath 206, thereby allowing the bristles 214 to make sufficient contact with a target tissue are during operation. To operate the bristles 214 of the brush 202, the shaft 220 may be moved back-and-forth (e.g., distally and proximally), perhaps controlled from the proximal end of the device (not shown), such that the bristles 214 rub against the target tissue area to collect a tissue sample.

A maximum outer diameter 224 of the distal tip 208 may be at least as large as the outer diameter 226 of the sheath 206 (e.g., at least at the distal end of the sheath 206). As shown, the maximum outer diameter 224 of the distal tip 208 is a cross-sectional dimension that extends in a direction that is perpendicular to the proximal direction. Advantageously, the distal tip 208 with these dimensions may seal the distal-end opening of the sheath 206 to prevent moisture, body tissue, and other unwanted materials from collecting inside the sheath 206 during deployment. Further, the distal tip 208 in the present embodiment includes a hemispherical shape on its distal side, which may displace fluid, tissue, and/or other particles from the desired pathway of the brush 202 as it is moved towards the target tissue area. Other shapes, such as the shape of the distal tip 208 of FIGS. 3-4 (listed as an example only), are also contemplated.

In FIGS. 5-6, the brush 202 includes a first tissue collection member 252 located on the proximal side of the distal tip 208. A second tissue collection member 254 (shown only in FIG. 6) is located within the sheath 206. Both of these tissue collection members are optional (and may be used with any embodiment consistent with the present description, where compatible), and they may be used in different locations in certain other embodiments. The first tissue collection member 252 and/or the second tissue collection member 254 may include any of the aspects (and variations thereof) discussed above with respect to tissue collection member 142 (of FIGS. 3-4), for example. Thus, the first tissue collection member 252 and/or the second tissue collection member 254 may collect and maintain tissue samples upon contact with the bristles 214 for later analysis by a medical professional.

Like the previous embodiments, the brush 202 may include at least one tracking device and/or radiopaque marker 250. For example, at least one tracking device and/or radiopaque marker 250 may be fixed to the shaft 220 (e.g., at the distal tip 208, the bristles 214, etc.), which may indicate the location of those components to a medical professional. Additionally, or alternatively, at least one tracking device and/or radiopaque marker may be fixed to the sheath 206 (not shown in this embodiment). Advantageously, by fixing at least one tracking device and/or radiopaque marker to the sheath 206 and at least one tracking device and/or radiopaque marker to the shaft 220, the location of each of the components of the brush 202 may be determined.

FIGS. 7-8 show another embodiment of a brush 302 in a first (deployment) state and a second (operational) state, respectively. This embodiment may include any of the features and advantages of the embodiments discussed above (and variations thereof), where compatible. In this embodiment, a sheath 306, a set of bristles 314, and a distal tip 308 are all movable relative to each other (but may otherwise be similar to previously-described corresponding components). For example, like previously-described embodiments, a first shaft 360 may be fixed to a set of bristles 314 and may be movable relative to the sheath 306 to move the bristles 314 during a tissue collection procedure. The distal tip 308 may be fixed to a second shaft 362, which may optionally be coaxial with the first shaft 360. While other configurations are also possible, the depicted first shaft 360 is hollow such that it includes an opening extending longitudinally therethrough, and the second shaft 362 extends through this opening. Thus, moving the second shaft 362 distally relative to the first shaft 360 causes the distal tip 308 to move distally relative to the bristles 314.

To operate the brush 302 (once it is deployed to an appropriate position), the distal tip 308 may move away from the sheath 306 to create working space 330 for the bristles 314 (either by moving the distal tip 308 distally, retracting the sheath 306 proximally, or both). Afterwards (or simultaneously), the first shaft 360 may be moved proximally relative to the distal tip 308 such that the bristles 314 are withdrawn from a cavity 316 of the distal tip 308. In other embodiments, the bristles 314 may initially be within the sheath 306, and therefore moving the bristles 314 into the working space 330 may occur when the bristles 314 are moved distally relative to the sheath 306. Once the bristles 314 are in the working space 330 (e.g., in an expanded state for suitable tissue contact), the bristles 314 may operate in accordance with the embodiments discussed above and variations thereof.

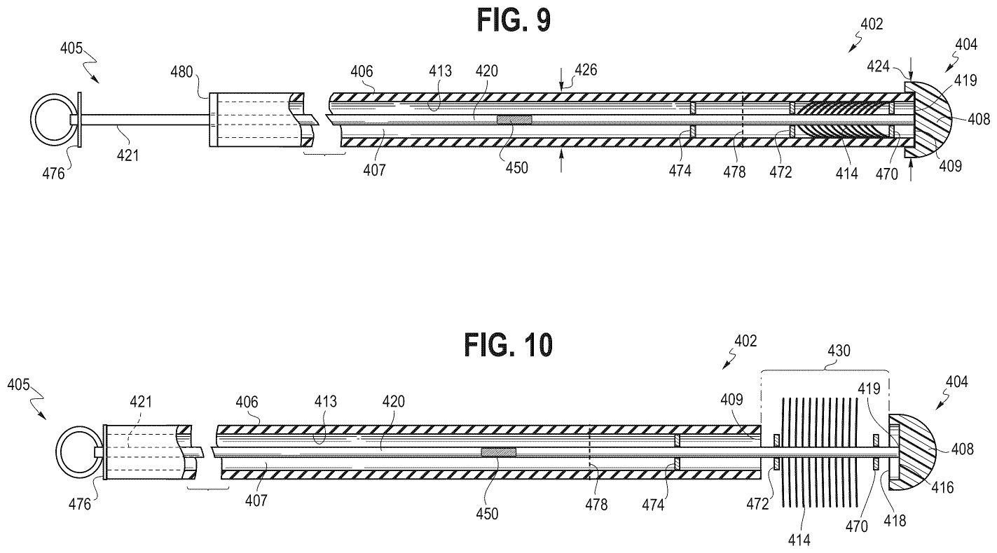

FIGS. 9-10 show another embodiment of a brush 402 in a first (deployment) state and a second (operational) state, respectively. This embodiment may include any of the features and advantages of the embodiments discussed above (and variations thereof), where compatible. In this embodiment, a shaft 420 extends through a cavity 407 of a sheath 406. The shaft 420 is moveable relative to the sheath 406. To transition from the first state (FIG. 9) to the second state (FIG. 10), the shaft 420 is moved distally relative to the sheath 406. To return the brush 402 to the first state from the second state, the shaft 420 is moved proximally relative to the sheath 406. While FIGS. 9-10 illustrate distal-proximal movement, the movement of the shaft 420 relative to the sheath 406 is not limited to this degree of freedom. For example, the shaft 420 may also rotate relative to the sheath 406.

A distal tip 408, alternatively referred to as a cap 408, is attached to a distal end 419 of the shaft 420. A set of bristles 414 is attached to the shaft 420 at a location proximal to the cap 408. And similar to other embodiments discussed above, a radiopaque marker 450 (e.g., a metallic, magnetic, and/or other suitable radiopaque material member or substance) may be included in or on the brush 402 at various locations, including on the shaft 420. At least two members 470 and 472 may also each be attached to the shaft 420 at locations proximal to the cap 408. The at least two members 470 and 472 are discussed in greater detail below.

The maximum diameter 424 of the cap 408 is greater than or equal to the outer diameter 426 of the sheath 406. The cap 408 includes an opening 418 that extends into a cavity 416 of the cap 408. The sizes of the opening 418 and the cavity 416 correspond to the diameter 426 of the sheath 406 such that the cap 408 is configured to seal a distal end 409 of the sheath 406 (e.g., FIG. 9, the first state). Further, with the opening 418 extending to the cavity 416 of the cap 408, the cap 408 removably receives the sheath 406. When the sheath 406 is received into the cavity 407 of the cap 408 (e.g., FIG. 9, the first state), sealing of the distal end 409 of the sheath 406 is improved as the surface-to-surface contact area between the cap 408 and the sheath 406 is greater. Advantageously, the cap 408 with these dimensions may seal the distal end 409 of the sheath 406 to prevent moisture, body tissue, other unwanted materials, and contamination from collecting inside the sheath 406 during deployment. Other features, such as an O-ring, are also contemplated within the cavity 416 of the cap 408 or near the distal end 409 of the sheath 406 to further improve sealing.

Similar to other embodiments, the cap 408 in the present embodiment includes a hemispherical shape on its distal side, which may displace fluid, tissue, and/or other particles from the desired pathway of the brush 402 as it is moved towards the target tissue area. Other shapes, such as the shape of the distal tip 108 of FIGS. 3-4 (listed as an example only), are also contemplated. A smooth, continuous, and/or rounded surface of the cap 408 helps to avoid damage to any surrounding tissue. And like other elements discussed herein, the cap 408 may be formed of silicon, plastic, or another suitable material.

As shown in FIGS. 9-10, the set of bristles 414 is attached to the shaft 420. Like other embodiments, the bristles 414 may include a tendency to expand when the brush 402 is moved to the second state (FIG. 10), and a brushing diameter of the bristles 414 may be greater than or equal to the diameter 426 of the sheath and/or the diameter 424 of the cap 408. In this embodiment, the shaft 420 is moved distally relative to the sheath 406 such that the set of bristles 414 exits the cavity 407 of the sheath 406 and expands outward in a working space 430 to collect a tissue sample from a target site (e.g., FIG. 10, the second state). While bristles 414 are depicted in the figures, the present invention is not limited to bristles 414. For example, in a non-exhaustive list, swabs, foams, fibers, absorbent material, and certain components with deformities or discontinuities may be used to collect the tissue sample from the target site at the working space 430.

At least two members 470 and 472 may also each be attached to the shaft 420 at locations proximal to the cap 408. A first member 470 may be attached at a location distal to the set of bristles 414 while still proximal to the cap 408. A second member 472 may be included at a location proximal to the set of bristles 414. As shown in FIG. 9, when the brush 402 is in the first state, the first member 470 and the second member 472 are located within the sheath 406. As shown in FIG. 10, the first member 470 and the second member 472 are located outside of the sheath 406 when the shaft 420 is moved distally relative to the sheath 406 to the second state.

The size of the at least two members 470 and 472 corresponds to the sheath 406. More specifically, an outer diameter and a shape of the at least two members 470 and 472 correspond to an inner diameter and an inner shape of the sheath 406 to form a tight fit within the cavity 407 of the sheath 406 against an inner surface 413 of the sheath 406. In this manner, the at least two members 470 and 472 are configured to seal the cavity 407 of the sheath 406 when the at least two members 470 and 472 are located within the sheath 406. The at least two members 470 and 472 may be substantially cylindrically and may have a central channel that is configured (e.g., sized and shaped) for receipt of the shaft 420. In a non-limiting example, the at least two members 470 and 472 may resemble a washer.

The at least two members 470 and 472 may be formed of silicon, plastic, rubber, or another suitable material. Further, it is contemplated that the at least two members 470 and 472 may be formed from a compressible material, or a material with a degree of softness to it, such that the central channel compresses around the shaft 420 when the at least two members 470 and 472 are located inside the sheath 406, which may provide a desirable friction on the shaft 420. Advantageously, such friction may enhance the feel to a medical professional and increase the precision of movement when operating the bristles 414 of the brush 402 (e.g., by adding a suitable amount of resistance to the shaft 420).

The at least two members 470 and 472 are also configured to wipe the inner surface 413 of the sheath 406 when the shaft 420 is moved proximally relative to the sheath 406 from the second state to the first state. Based upon the first member 470 and the second member 472 being formed (e.g., shaped and sized) to fit the cavity 407 of the sheath 406, the first member 470 and the second member 472 may wipe against the inner surface 413 of the sheath 406 when the shaft 420 is retracted proximally into the sheath 406. Advantageously, the wiping of the inner surface 413 of the sheath 406 via the first member 470 and the second member 472 helps reduce moisture, body tissue, other unwanted materials, and contamination that may have collected inside the sheath 406 while the brush 402 was in its second state during sample collection. Reducing such contamination helps improve the accuracy of a biopsy. Further, any such fluid or material that may have entered the cavity 407 of the sheath 406 during tissue collection is moved proximally in the cavity 407 via the second member 472. Advantageously, any possible contamination and/or excess fluid may remain proximal to the second member 472 and away from the set of bristles 414 and a collected sample, which would be distal to the second member 472 post-collection.

In addition to the at least two members 470 and 472, the brush may further include a third member 474. The third member 474 may have a similar shape, size, and construction as the first member 470 and the second member 472. As shown in FIGS. 9-10, the third member 474 may be attached to the shaft 420 at a location proximal to the second member 472. Further, as shown in FIG. 10, when the shaft 420 is moved distally relative to the sheath 406 to the second state, the third member 474 remains located within the sheath 406, unlike the first member 470 and the second member 472. Similar to the at least two members 470 and 472, the third member 474 is located within the sheath 406 in the first state and is configured to seal the cavity 407 of the sheath 406 when located within the sheath 406. For the third member 474, this again includes in the second state. Beneficially, the sealing of the cavity 407 of the sheath 406 via the third member 474 reduces moisture, body tissue, other unwanted materials, and contamination from collecting inside the sheath 406, including when the brush 402 is in the second state and the first member 470 and the second member 472 are located outside of the sheath 406.

To prevent the third member 474 from exiting the cavity 407 of the sheath 406 when the shaft 420 moves distally from the first state to the second state, a protrusion 476 may be included towards a proximal end 405 of the brush 402. The protrusion 476 may be attached to the shaft 420 towards a proximal end of the shaft 420. In a non-limiting example, the protrusion 476 may have a larger radius in one portion or a larger diameter than the sheath 406, such that the shaft 420 cannot move further in the distal direction once the protrusion 476 contacts the sheath 406. In another example, the protrusion 476 may be located within the cavity 407 of the sheath 406 and may contact another structure within the sheath 406 to prevent further distal movement of the shaft 420 relative to the sheath 406.

As shown in FIGS. 9-10, the brush 402 may also include a locking mechanism 480 near its proximal end 405. The locking mechanism 480, when in a locked state, prevents the shaft 420 from moving relative to the sheath 406. The locking mechanism 480 may remain in the locked state during deployment of the brush 402 to the target site (e.g., FIG. 9). Once at the target site, the locking mechanism 480 may be removed or changed to an unlocked state, allowing movement of the shaft 420 relative to the sheath 406 (e.g., FIG. 10). In a non-exhaustive list of exemplary locking mechanisms 480, such locking mechanisms 480 may include a removable pin inserted through a hole in the shaft or a clamp. While not shown in FIGS. 9-10, the brush 402 may include additional features at its proximal end 405 for controlling adjustment from the first state to the second state, and/or for controlling the working features at the distal end 404 of the brush 402. Advantageously, the working components on the proximal end 405 of the brush 402 may be operated from a location outside the patient body, thereby providing a procedure that is relatively non-invasive or minimally invasive.

Additionally, a cutline 478 may be included with the brush 402. After the tissue sample has been collected from the target site, the brush 402 has been returned to the first state from the second state, and the brush 402 has been withdrawn from a patient's body, the tissue sample and set of bristles 414 reside within the cavity 407 of the sheath 406 between the first member 470 and the second member 472. To retrieve the tissue sample, the cutline 478 may be located on the sheath 406 proximal to the second member 472. Advantageously, by cutting the brush 402 at this location proximal to the second member, a quasi-container is formed between the first member 470, the second member 472, and the sheath 406 that protects the tissue sample from further contamination while also being easier to handle. It is also contemplated that a second cutline may be included distal to the first member 470 and proximal to the cap 408.

Similar to the other embodiments, the embodiment depicted in FIGS. 9-10 is configured to be used within an endoscopic device. The brush 402 (e.g., the shaft 420 and the sheath 406) has sufficient flexibility to be used within endoscopic devices and to navigate through curves within the patient's body.

To operate the brush 402, the brush 402 is delivered in the first state to the target location. During deployment, the cap 408 seals the distal end 409 of the sheath 406. The first member 470 also seals the cavity 407 of the sheath 406. At the target location and if present, the locking mechanism may then be removed, or changed from the locked state to the unlocked state, to allow movement of the shaft 420 relative to the sheath 406. The brush 402 is moved to the second state via the shaft 420 moving distally relative to the sheath 406. In the second state, the brush 402 may collect the tissue sample from the target site via the set of bristles 414 in the working space 430 contacting and/or rubbing against the tissue at the target location. Collecting the tissue sample may include moving the shaft 420 between the proximal direction and the distal direction (e.g., back-and-forth) at the target location. Additionally, the shaft 420 may be rotated at the target location to help collect the tissue sample at the set of bristles 414. While in the second state and if present, the third member seals the cavity 407 of the sheath 406. The protrusion 476 may also be included to prevent further distal movement of the shaft 420 relative to the sheath 406, which would cause the third member 474 to exit the sheath 406. Once the tissue sample has been collected, the brush 402 may be returned to the first state via the shaft 420 moving proximally relative to the sheath 406. During the proximal movement of the shaft 420, the second member 472 wipes the inner surface 413 of the sheath 406 to reduce possible contamination within the sheath. The second member 472 may then also form a seal within the cavity 407 of the sheath 406 to prevent possible fluid and/or contamination that entered the cavity 407 between the third member 474 and the second member 472 from affecting the tissue sample at the set of bristles 414. The first member 470 also seals the tissue sample at the set of bristles 414 within the cavity 407 of the sheath 406 in the first state. In the first state and with the tissue sample collected, the cap 408 once more seals the distal end 409 of sheath 406. The brush 402 may be removed from the target location and the patient's body. If present, the brush 402 may be cut at the cutline 478 located proximal to the second member. Throughout the use of the brush 402, the radiopaque marker 450 may be used to indicate the location of the brush 402 during the procedure.

While various embodiments have been described, it will be apparent to those of ordinary skill in the art that many more embodiments and implementations are possible. Accordingly, the embodiments described herein are examples, not the only possible embodiments and implementations.

The subject-matter of the disclosure may also relate, among others, to the following aspects:

A first aspect involves an embodiment of a brush for biopsy and variations thereof. Without limitation, the brush may include one or more of the following features: a sheath, a shaft that extends through a cavity of the sheath, the shaft is moveable relative to the sheath, a cap located at a distal end of the shaft, the cap is configured to seal a distal end of the sheath, a set of bristles attached to the shaft, at least two members attached to the shaft at locations proximal to the cap, and the at least two members are configured to seal the cavity when located within the sheath. Further, the brush may include a first member of the at least two members attached to the shaft at a location distal to the set of bristles and a second member of the at least two members attached to the shaft at a location proximal to the set of bristles.

In a second aspect, the brush of the first aspect may further include the first member and the second member located outside of the sheath when the shaft is moved distal to the sheath.

In a third aspect, the brush of the second aspect may additionally include the first member and the second member configured to wipe an inner surface of the sheath when the shaft is moved proximal to the sheath.

In a fourth aspect, the brush of any of the first through third aspects may also include a third member of the at least two members attached to the shaft at a location proximal to the second member.

In a fifth aspect, the brush of the fourth aspect may further include the first member and the second member located outside of the sheath and the third member located within the sheath when the shaft is moved distal to the sheath.

In a sixth aspect, the brush of the fifth aspect may additionally include a protrusion located towards a proximal end of the shaft. The protrusion may be configured to prevent the third member from exiting the sheath when the shaft is moved distal to the sheath.

In a seventh aspect, the brush of any of the first through sixth aspects may also include at least one radiopaque marker within or on the brush that is configured to indicate a location of the at least one radiopaque marker within the brush.

In an eighth aspect, the brush of any of the first through seventh aspects may further include the sheath having a cutline located proximal to the second member when the first member and the second member are located within the sheath.

In a ninth aspect, the brush of any of the first through eighth aspects may additionally include a locking mechanism located at a proximal end of the brush. The locking mechanism may be configured to prevent the shaft from moving relative to the sheath when the locking mechanism is in a locked state.

In a tenth aspect, the brush of any of the first through ninth aspects may further include the brush configured to be used within an endoscopic device.

An eleventh aspect involves another embodiment of a brush for biopsy. Without limitation, the brush may include the following features: a sheath, a shaft that extends through a cavity of the sheath, the shaft is moveable relative to the sheath, a set of bristles attached to the shaft, a first member attached to the shaft distal to the set of bristles, the first member configured to seal the cavity when it is located within the sheath, a second member attached to the shaft proximal to the set of bristles, and the second member configured to seal the cavity when located within the sheath. Additionally, the brush may include the first member and the second member located within the sheath in a first state. The brush may also include the first member, the set of bristles, and the second member located outside of the sheath in a second state when the shaft is moved distal to the sheath.

In a twelfth aspect, the brush of the eleventh aspect may further include the second member configured to wipe an inner surface of the sheath when the brush moves in a proximal direction from the second state to the first state.

In a thirteenth aspect, the brush of the twelfth aspect may additionally include a third member attached to the shaft at a location proximal to the second member. The third member may be configured to seal the cavity when located within the sheath. The third member may also be located within the sheath in the first state and the second state.

In a fourteenth aspect, the brush of the thirteenth aspect may further include protrusion located towards a proximal end of the shaft. The protrusion may be configured to prevent the third member from exiting the sheath when the shaft moves in a distal direction from the first state to the second state.

In a fifteenth aspect, the brush of any of the eleventh through fourteenth aspects may additionally include a cap at a distal end of the shaft. The sheath may be received into the cap in the first state. The shaft may also be configured to seal the cavity in the first state.

In a sixteenth aspect, the brush of any of the eleventh through fifteenth aspects may further include at least one radiopaque marker within or on the brush that is configured to indicate a location of the at least one radiopaque marker within the brush.

In a seventeenth aspect, the brush of any of the eleventh through sixteenth aspects may additionally include the sheath having a cutline proximal to the second member when in the first state.

An eighteenth aspect involves an embodiment of a method of using a brush for biopsy. Without limitation, the method may include delivering the brush in a first state to a target location, where the brush may include a shaft attached to a set of bristles, a first member distal to the set of bristles, and a second member proximal to the set of bristles, and where the first state may include the first member, the set of bristles, and the second member located within a sheath that is moveable relative to the shaft. The method may also include moving the shaft in a distal direction to a second state where the first member, the set of bristles, and the second member are located outside of the sheath. The method may further include collecting a tissue sample and moving the shaft in a proximal direction to the first state, where the second member is configured to wipe an interior surface of the sheath, and where the first member is configured to seal a cavity of the sheath.

In a nineteenth aspect, collecting the tissue sample of the method of the eighteenth aspect may additionally include moving the shaft between the proximal direction and the distal direction.

In a twentieth aspect, the method of any of the eighteenth through nineteenth aspects may also include removing the brush from the target location and cutting the brush at a cutline located proximal to the second member when the brush is in the first state.

In alternative embodiments, a first aspect involves an embodiment of a brush for non-invasive biopsy, and variations thereof. Without limitation, the brush may include one or more of the following features: a distal tip forming a cavity, where an opening of the cavity faces proximally; a shaft that is movable relative to the distal tip, where a distal end of the shaft is located within the cavity when the brush is in a first state, and where at least a portion of the distal end of the shaft is located outside the cavity when the brush is in a second state; and a set of bristles located at the distal end of the shaft, where the bristles at least partially move from within the cavity of the distal tip to outside the cavity of the distal tip when the brush moves from the first state to the second state.

In a second aspect, the brush of the first aspect may further include an outer sheath, where the shaft extends through the outer sheath, and where a maximum diameter of the distal tip is at least as large as an outer diameter of the outer sheath.

In a third aspect, the brush of the second aspect may be configured such that the distal tip may at least partially seals a distal-end opening of the outer sheath when the brush is in the first state.

In a fourth aspect, the brush of any of the second through third aspects may further include a plug located at a distal end of the outer sheath, where the plug at least partially seals a distal-end opening of the outer sheath, and where the shaft extends through an opening of the plug.

In a fifth aspect, the brush of the fourth aspect may be configured such that at least a portion of the plug retracts into the outer sheath when the brush moves from the first state to the second state.

In a sixth aspect, the brush of the fifth aspect may be configured such that a projection fixed to the shaft is configured to contact a distal surface of the plug to provide a force for retracting the plug into the outer sheath when the brush moves from the first state to the second state.

In a seventh aspect, the brush of any of the second through sixth aspects may be configured such that the bristles include a tendency to expand to a bristle diameter when the brush is in the second state, and where the bristle diameter is larger the maximum diameter of the distal tip and the outer diameter of the outer sheath.

In an eighth aspect, the brush of any of the second through seventh aspects may be configured such that a tissue collection member is located inside the outer sheath, where the tissue collection member includes an absorbent material configured to collect and retain a tissue sample upon contact with at least one of the bristles.

In a ninth aspect, the brush of any of the first through eighth aspects may be configured such that the distal tip includes tissue collection member located inside the cavity, where the tissue collection member includes an absorbent material configured to collect and retain a tissue sample upon contact with at least one of the bristles.

In a tenth aspect, the brush of any of the first through ninth aspects may be configured such that the distal tip includes a taper having an apex at a distal terminus of the brush.

An eleventh aspect involves another embodiment of a brush for non-invasive biopsy. Without limitation, the brush may include the following features: a distal tip located at a distal end of a shaft, where a set of bristles is located on the shaft at a location that is proximal of the distal tip; and an outer sheath that is movable in a proximal direction relative to the distal tip, where the bristles are at least partially located in the outer sheath when the brush is in a first state, where the bristles include a tendency to expand to a brushing diameter when the outer sheath is in a second state, and where the brushing diameter is larger than an outer diameter of the outer sheath.

In a twelfth aspect, the brush of the eleventh aspect is configured such that an outer diameter of the distal tip is at least as large of the outer diameter of the outer sheath, and where the outer diameter of the distal tip extends in a direction that is perpendicular to the proximal direction.

In a thirteenth aspect, the brush of any of the eleventh through twelfth aspects is configured such that the distal tip at least partially seals a distal opening of the outer sheath when the brush is in the first state.

In a fourteenth aspect, the brush of any of the eleventh through thirteenth aspects is configured such that the distal tip includes a magnetic and/or metallic radiopaque marker and/or tracking device configured to indicate a location of the distal tip

In a fifteenth aspect, the brush of any of the eleventh through fourteenth aspects is configured such that a tissue collection member is located inside the outer sheath, where the tissue collection member includes an absorbent material configured to collect and retain a tissue sample upon contact with at least one of the bristles.

In a sixteenth aspect, the brush of any of the eleventh through fifteenth aspects is configured such that the distal tip includes tissue collection member, where the tissue collection member includes an absorbent material configured to collect and retain a tissue sample upon contact with at least one of the bristles.

A seventeenth aspect involves another embodiment of a brush for non-invasive biopsy. Without limitation, the brush may include one or more of the following features: a first shaft for controlling a position of a set of bristles, where the set of bristles is fixed relative to the first shaft; a second shaft for controlling a position of a distal tip of the brush, where the distal tip is fixed relative to the second shaft, and where the distal tip forms a distal terminus of the brush; and an outer sheath, where the first shaft extends through the outer sheath, and where the second shaft extends through the outer sheath, where the first shaft and the second shaft are each movable relative to the outer sheath such that the bristles are movable relative to the outer sheath and the distal tip is movable relative to the outer sheath.

In an eighteenth aspect, the brush of the seventeenth aspect is configured such that the distal tip incudes a cavity for receiving at least one bristle of the set of bristles.

In a nineteenth aspect, the brush of any of the seventeenth through eighteenth aspects is configured such that a tissue collection member is located inside the cavity of the distal tip, where the tissue collection member includes an absorbent material configured to collect and retain a tissue sample upon contact with at least one of the bristles.

In a twentieth aspect, the brush of the seventeenth aspect is configured such that the first shaft and the second shaft are coaxial.

In addition to the features mentioned in each of the independent aspects enumerated above, some examples may show, alone or in combination, the optional features mentioned in the dependent aspects and/or as disclosed in the description above and shown in the figures.

We claim:

1. A brush for biopsy, comprising:
a sheath;
a shaft extending through a cavity of the sheath, the shaft being moveable relative to the sheath;
a cap located at a distal end of the shaft, the cap being configured to seal a distal end of the sheath;
a set of bristles attached to the shaft; and
at least two members attached to the shaft at locations proximal to the cap, the at least two members being configured to seal the cavity when located within the sheath,
wherein a first member of the at least two members is attached to the shaft at a location distal to the set of bristles and a second member of the at least two members is attached to the shaft at a location proximal to the set of bristles.

2. The brush of claim 1, wherein the first member and the second member are located outside of the sheath when the shaft is moved distal to the sheath.

3. The brush of claim 2, wherein the first member and the second member are configured to wipe an inner surface of the sheath when the shaft is moved proximal to the sheath.

4. The brush of claim 1, comprising a third member of the at least two members attached to the shaft at a location proximal to the second member.

5. The brush of claim 4, wherein the first member and the second member are located outside of the sheath and the third member is located within the sheath when the shaft is moved distal to the sheath.

6. The brush of claim 5, comprising a protrusion located towards a proximal end of the shaft, the protrusion being configured to prevent the third member from exiting the sheath when the shaft is moved distal to the sheath.

7. The brush of claim 1, comprising at least one radiopaque marker within the brush, the at least one radiopaque marker being configured to indicate a location of the at least one radiopaque marker within the brush.

8. The brush of claim 1, the sheath comprising a cutline located proximal to the second member when the first member and the second member are located within the sheath.

9. The brush of claim 1, comprising a locking mechanism located at a proximal end of the brush, the locking mechanism being configured to prevent the shaft from moving relative to the sheath when the locking mechanism is in a locked state.

10. The brush of claim 1, wherein the brush is configured to be used within an endoscopic device.

11. A brush for biopsy, comprising:
a sheath;
a shaft extending through a cavity of the sheath, the shaft being moveable relative to the sheath;
a set of bristles attached to the shaft;
a first member attached to the shaft distal to the set of bristles, the first member being configured to seal the cavity when located within the sheath; and
a second member attached to the shaft proximal to the set of bristles, the second member being configured to seal the cavity when located within the sheath,
wherein the first member and the second member are located within the sheath in a first state, and
wherein the first member, the set of bristles, and the second member are located outside of the sheath in a second state when the shaft is moved distal to the sheath.

12. The brush of claim 11, wherein the second member is configured to wipe an inner surface of the sheath when the brush moves in a proximal direction from the second state to the first state.

13. The brush of claim 12, comprising a third member attached to the shaft at a location proximal to the second member, wherein the third member is configured to seal the cavity when located within the sheath, and wherein the third member is located within the sheath in the first state and the second state.

14. The brush of claim 13, comprising a protrusion located towards a proximal end of the shaft, the protrusion being configured to prevent the third member from exiting the sheath when the shaft moves in a distal direction from the first state to the second state.

15. The brush of claim 11, comprising a cap at a distal end of the shaft, wherein the sheath is received into the cap in the first state, and wherein the cap is configured to seal the cavity in the first state.

16. The brush of claim 11, comprising at least one radiopaque marker within the brush, the at least one radiopaque marker being configured to indicate a location of the at least one radiopaque marker within the brush.

17. The brush of claim 11, the sheath comprising a cutline proximal to the second member when in the first state.

18. A method of using a brush for biopsy, comprising:
delivering the brush in a first state to a target location, the brush comprising a shaft attached to a set of bristles, a first member distal to the set of bristles, and a second member proximal to the set of bristles, wherein the first state includes the first member, the set of bristles, and the second member located within a sheath that is moveable relative to the shaft;
moving the shaft in a distal direction to a second state, wherein the first member, the set of bristles, and the second member are located outside of the sheath;
collecting a tissue sample; and
moving the shaft in a proximal direction to the first state, wherein the second member is configured to wipe an interior surface of the sheath, and wherein the first member is configured to seal a cavity of the sheath.

19. The method of claim 18, wherein collecting the tissue sample includes moving the shaft between the proximal direction and the distal direction.

20. The method of claim 18, comprising:
removing the brush from the target location; and
cutting the brush at a cutline located proximal to the second member when the brush is in the first state.
